SEQUENCE LISTING (7 sequences)

SEQ ID NO: 1 - DNA, 32 bp, Artificial
  synthetic ologonucleotide primer
ataacaatttcacacaggaaacagctatgacc

SEQ ID NO: 2 - DNA, 32 bp, Artificial
  synthetic oligonucleotide primer
cagggttttcccagtcacgacgttgtaaaacg

SEQ ID NO: 3 - DNA, 32 bp, Artificial
  synthetic oligonucleotide primer
aaaaaaaaaccatgtctccggatctctggtac

SEQ ID NO: 4 - DNA, 31 bp, Artificial
  synthetic oligonucleotide primer
cagctcgagctcctttgaaagcaccctaaac

SEQ ID NO: 5 - protein, 11 aa, Human immunodeficiency virus
YGRKKRRQRRR

SEQ ID NO: 6 - protein, 11 aa, Artificial
  synthetic TAT sequence optimized by random mutagenisis
YARAAARQARA

SEQ ID NO: 7 - protein, 313 aa, Homo sapiens
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDYSGQGVDQLQRVIDTIKTNPDDRRIIMCAWNPRDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

(19) United States
(12) Patent Application Publication
(10) Pub. No.: US 2008/0045451 A1
(43) Pub. Date: Feb. 21, 2008

(54) Novel thymidylate synthase mutants

(76) Inventors: Lawrence Loeb, Bellevue, WA (US); Werner Geurtsen, Bainbridge, WA (US); Ray Monnat, Seattle, WA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/558,246

(22) Filed: Nov. 9, 2006

Related U.S. Application Data
(63) Continuation-in-part of application No. 10/308,192, filed on Dec. 3, 2002, now abandoned.
(63) Continuation-in-part of application No. 10/450,629, filed on Nov. 20, 2003, now abandoned.
(60) Provisional application No. 60/334,557, filed on Dec. 3, 2001.

(30) Foreign Application Priority Data
Jun. 29, 2002 (EP) 02014489.5
Dec. 15, 2000 (DE) 10062766.8

(51) Int. Cl. A61K 31/70; A61P 43/00; C07H 21/04; C07K 16/00; C12N 15/00; C12N 5/00

(52) U.S. Cl. 514/12; 435/320.1; 435/325; 514/44; 530/350; 536/23.2

(57) ABSTRACT

Novel thymidylate synthase (TS mutants) are disclosed differing from human wild type thymidylate synthase in single, double, or multiple mutations, which show intact enzyme activity and enhanced resistance to TS-inhibiting drugs like 5-fluorouracil or 5-fluoro-2-deoxyuridinemonophosphate. All these mutants can be used for the protection of normal human cell populations against the toxic manifestation of analogs that inhibited TS. Drugs for the local treatment or prevention of a mucositis in the oral cavity and the gastrointestinal tract caused by chemotherapy with TS-inhibitors are also provided.

### CROSS-REFERENCE TO RELATED APPLICATIONS

This application is a continuation-in-part of and claims benefit of co-pending U.S. patent application Ser. No. 10/308,192, filed Dec. 3, 2002, which in turn claims benefit of U.S. provisional patent application No. 60/334,557, filed Dec. 3, 2001 and European patent application 02014489.5, filed Jun. 29, 2002. This application is also a continuation-in-part of and claims benefit of co-pending U.S. patent application Ser. No. 10/450,629, filed Nov. 20, 2003, which in turn claims benefit of International patent application PCT/DE01/04864, filed Dec. 17, 2001, and German patent application 100 62 766.8, filed Dec. 15, 2000. The complete contents of each of these applications are hereby incorporated by reference.

### STATEMENT REGARDING FEDERALLY FUNDED RESEARCH

This invention was made using funds from a grant from the National Institutes of Health having grant number 1RO1 CA78885. The United States government may have certain rights in this invention.

### FIELD OF THE INVENTION

The invention is related to novel human thymidylate synthase mutants (thymidylate synthase mutants, TS mutants) and DNA encoding these mutants as well as the use thereof, especially in a pharmaceutical composition.

### BACKGROUND OF THE INVENTION

According to figures of the American Cancer Society new cases of cancer of the colon and of the breast, respectively, in the United States in 1997 had been estimated to 130,000 and 180,000 with 55,000 and 66,000 deaths, respectively, due to these cancers (American Cancer Society, Cancer Facts and Figures 151; 1997).

Thymidylate synthase (TS) has been known for a long period of time and has been described for instance by Carreras et al (Carreras and Santi D V. 1995). The amino acid sequence of TS is depicted in FIG. 8. TS is a 72 kD protein that catalyzes the methylation of dUMP to dTMP. In eukaryotic cells, DNA synthesis is dependent on the production of dTMP by this de novo pathway (Komberg and Baker, DNA replication, 1992). In addition, it is known that TS activity is greatest in rapidly proliferating cells (Rode et al., JBC 225:1305, 1980). For these reasons, TS has been an important target for the design of chemotherapeutic agents. Various chemotherapeutic drugs, e.g., 5-fluorouracil (5-FU) or 5-fluoro-2-deoxyuridine monophosphate (FdUMP) inhibit thymidylate synthase and as a result interfere with the DNA-metabolism of tumor cells, such as squamous cell carcinomas in the gastrointestinal area or breast tumors. 5-Fluorouracil (5-FU) is used extensively for the treatment of carcinoma of the breast and colon, and is effective either as a single agent or in combination with other drugs in ablation of these tumors.

Unfortunately, the inhibition of TS is not specific to tumors but also occurs in normal cells. Thus, TS inhibitors cannot be administered well-aimed in a sufficient manner, so that healthy cells are also damaged, which then causes severe side effects, limiting their effective dosage. For example, the toxicity to bone marrow is manifested by anemia, leukopenia, and thombocytopenia. In addition, a mucositis of the gastrointestinal tract including the oral cavity is frequently associated with chemotherapy using TS inhibitors. The effects of a mucositis can be so severe that the chemotherapy has to be stopped or shortened or the optimum dosage of the chemotherapy has to be reduced, which impairs the prognosis of the cancer therapy. Additionally, more infections are associated with a damaged mucosa, which cause an increased morbidity, dietary problems and a drastically reduced quality of life of the patients. Presently, such a mucositis can be only treated symptomatically with little success.

Due to the importance of TS inhibitors in cancer therapy there is an urgent need for new resistant TS mutants which can be used to protect normal human cell populations against the toxic manifestation of analogs that inhibit TS.

A human thymidylate synthase, which is mutated at amino acid residues 49, 52, 108, 221, and 225 was disclosed in WO 98/33518. The aim of the authors is to understand the blocking and resistance mechanisms using specific mutations of the thymidylate synthase based on the knowledge of its three dimensional structure, in order to develop new and better cancer drugs for the chemotherapy with TS inhibitors. For this, mutants using random and site directed mutation were produced, isolated, and characterized. It is also mentioned that the mutants may be potentially usable for gene therapy, in order to protect specific normal cells or to serve as selection marker in therapeutic gene transfer protocols. However, no usable gene therapeutic procedure is described.

Human TS mutants have been also reported, which are resistant to Thymitaq™ (AG337) and 5-fluoro-2-deoxyuridylate. Such mutants were investigated for instance within the scope of mechanistic studies about the DNA synthesis (Tong Y, et. al. 1998. J Biol Chem. 273: 11611-11618).

### SUMMARY OF THE INVENTION

The object of the invention is to generate novel thymidylate synthase enzymes that produce enhanced resistance to 5-fluorouracil (5-FU), 5-fluorouridine (5-FdUR), and related drugs, and to make them available for therapeutic use.

According to this invention there are now provided a series of mutant human thymidylate synthase enzymes which show high resistance to the drug 5-FU and related analogs. These mutant proteins and respective gene constructs are useful in transforming human non-tumorigenic cells (mucosal cells, bone marrow cells, etc.) either in vitro or in vivo prior or during the treatment of a patient receiving chemotherapy with TS-inhibiting drugs due to cancer or other diseases. The presence of these transformed cells will reduce the clinically severe myelo-suppression or alteration in gastrointestinal cells observed during treatment with the aforementioned drugs, as well as allow augmentation of the dose of chemotherapeutics to be implemented, since severe side effects due to this chemotherapy are reduced or eliminated.

The invention also provides a drug for the local treatment or prevention of a mucositis, which is induced by a chemotherapy with inhibitors of thymidylate synthase (in the following abbreviated as TS), specifically in the gastrointestinal tract including the oral cavity, and other adverse effects due to TS inhibitors, which can be treated locally. Generally, the invention concerns the application of specific TS mutants for this purpose.

### BRIEF DESCRIPTION OF THE DRAWINGS

FIG. 1. Number of amino acid substitutions. A. Non-selected mutants; B. active mutants; C. 5-FdUR-resistant mutants. Unselected mutants were obtained from transformed bacterium grown in rich medium. Active mutants were obtained from bacteria grown on M9 minimal medium. 5-FdUR-resistant mutants were collected from cells grown on M9 medium containing 5-FdUR (200 nM or 300 nM). To confirm whether the plasmid DNA recovered from E. coli cells leads to the resistance to 5-FdUR, the recovered plasmid DNA was retransformed into fresh χ2913 cells and the survival experiments were performed using varying amounts of 5-FdUR. Forty-three clones showing resistance to 5-FdUR in the second survival experiment were selected. The 630 bases, constituting two-thirds of the TS gene, were sequenced. Since the full length of TS cDNA is 939 bases long, 1.5 times as many substitutions are expected to be present throughout the protein.

FIG. 2. Distribution of amino acid substitutions in 5-FdUR-resistant TS mutants. All of the amino acid substitutions detected in forty-one 5-FdUR-resistant mutants were presented. The amino acid residues of wild type TS protein were shown under the line. A double underlining indicates residues absolutely conserved among all TS proteins reported (Carreras and Santi, 1995). A single underlining indicates residues that are conserved in more than 50% of the TS proteins. Non-conserved residues are not underlined. The percent conservation was conservation was calculated by comparing thymidine syntheses spanning viruses to mammals (Carreras and Santi, 1995). Spiral shapes designate α-helices and arrows are β-sheets.

FIG. 3. Survival of 5-FdUR-resistant TS mutants. E. coli 2913 cells expressing wild type or mutant TS protein were grown on M9 minimal plates containing 5-FdUR for 48 h. Survival was determined by counting colonies at each dose of 5-FdUR and is expressed as a fraction of the survival of untreated cells. A. 5-FdUR-resistant mutants isolated in the present and previous experiments. Mutant 302, the most resistant clone in this library, carries two amino acids substitutions, T53S and Y258F. Survival is compared to that of the quadruple mutant T51S;K82Q;K99D;N171S (Mutant 362), the double mutant T51S; G52S, the most resistant in previous experiments; and the wild type. (B) Generation of single mutants. Mutant 302, carries two amino acid substitutions, T53S and Y258F. Each single mutant was made and the survival curve was compared to wild type and the parent mutant. (T53S was less resistant than Y258F. In this figure, the curve for T53S is that for T51S. The result was shown on p141 (Feb. 8, 2001). C. Mutants carrying amino acid substitutions at Asp254 residue. Four amino acid substitutions were accumulated at Asp254 residue (three of four substitutions were Asp to Glu changes and the other was an Asp to Asn substitution) Three different single mutants at Asp254 residue were created (D254E, D254N and D254A). Survival curves of these single mutants and mutant 318, carrying three amino acid substitutions including D254E, were compared.

FIG. 4. Location of amino acid substitutions in single 5-FdUR-resistant mutants in the three-dimensional structure of human TS. A ribbon drawing of one subunit from the 1.9 Å structure of 32 dimeric human TS complexed with dUMP and ralitrexed, an antifolate drug, is shown, with the dimer interface furthest from the viewer. Residues 125 are disordered and are not in the crystallographic model; N and C represent the amino and carboxyl termini of residues 26313, respectively. The essential, active site Cys195 is represented by a green ball at its alpha carbon position. The ligands are shown in purple. All of the published amino acid substitutions in single, 5-FdUR-resistant mutants are mapped. The red balls denote PCR-generated mutations reported here; the blue bells denote mutations generated in this study by site-directed mutagenesis and mutations previously reported from this laboratory; the yellow balls denote mutations described by others. The coordinate set for the ternary complex was obtained from Protein Data Bank; chain 1HVY:A is modeled. The drawing, generously provided by Dr. Elinor Adman, was made by using the programs Molscript and Raster 3D.

FIG. 5. Graph showing the 5-FdUR (5-fluorodeoxyuridine=active metabolite, which is generated of 5-fluorouracil in the cells) resistance of wild type TS (TS_{WT}) compared to a TS mutant, where in position 254 the amino acid residue D (aspartic acid) was replaced by E (glutamic acid) (TS_{D254E}). The y-axis shows the survival rate of the bacteria in %, which were transfected with a TS-expression vector, referred to TS_{WT} transfected bacteria in the absence of the TS-inhibitor 5-FdUR. The x-axis shows the applied concentrations of 5-FdUR.

FIG. 6. Graph showing the survival rate in % of 3T3 fibroblasts incubated with 2.5 μM 5-FU (5-fluorouracil) after 24 h dependent on the presence or absence of 200 ng!ml of the TS mutant TS_{D254E}.

FIG. 7. Graph showing the survival rate of primary human gingival fibroblasts in % in the presence of 10 mM 5-FU after 24 h, either in presence or absence of 40 ng/ml of TS mutant TS_{D254E}.

FIG. 8. The amino acid sequence of TS (SEQ ID NO: 7)

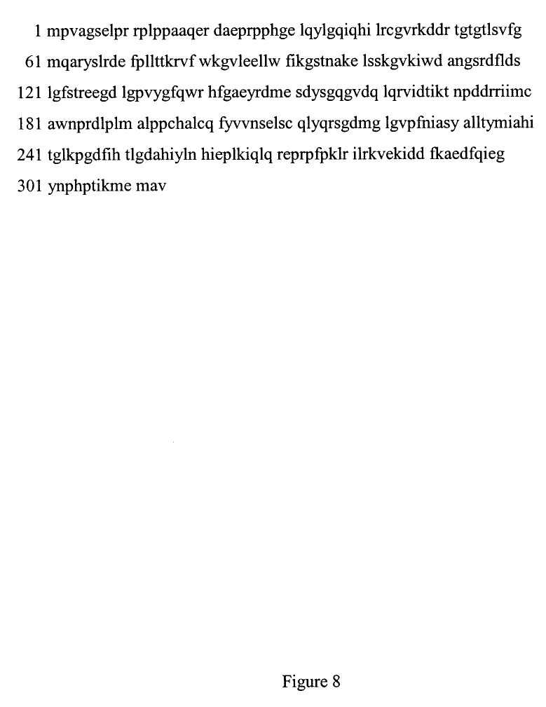

### DETAILED DESCRIPTION OF THE PREFERRED EMBODIMENTS OF THE INVENTION

The following human thymidylate synthase enzymes with single, double or multiple mutations are part of this invention:

Thymidylate synthase mutants, differing from human wildtype thymidylate synthase in single mutations at positions: E23, T53, V84, K93, D110, D116, M190, P194, S206, M219, H250, D254, Y258 or K284, where further silent or non-functional mutations with respect to TS activity are not excluded.

Preferred single mutations of the thymidylate synthase mutants cited above are E23G, D48V, T51S, T53S, V84A, K93E, D110E, D116A, M19OL, P194Q, S206G, M219V, H250L, D254E, D254A, D254N, Y258S, Y258F or K284N.

Thymidylate synthase mutants differing from human wildtype thymidylate synthase in double or multiple mutations at positions:
55, 106, and 284;
5,78, and 219;
45 and 254;
193 and 231;
6,69, and 211;
5,13, and 231;
103 and 204;
142 and 225;
17,116,and 254;
117, 169, and 254;
120 and 278;
38 and 104;
8, 81, 131, and 230;
51, 82, 99, and 171;
167 and 192,
   where further silent or non-functional mutations with respect to TS activity are not excluded. Preferred double or multiple mutations are:
   T55I, V106A, and K284I;
   G5S, R78C, and M219I;
   V45A and D254N;
   P193S and A231G;
   S6N, D69Q, and Q211L;
   G5D, L13R, and A231T;
   S103T and V204A;
   F142S and F225I;
   A17T, D116A, and D254E;
   F117S, K169R, and D254E;
   S120T and K278R;
   Q38H and K104D;
   L8Q, W81G, L131V, and Y230F;
   T51S, K82Q, K99D, and N171S;
   T167S and L192P.

Unexpectedly numerous mutants have been found where the position of the mutation(s) is(are) quite remote from the center of activity of TS, especially remote from the Arg50 loop. Mutations throughout the sequence have been shown to influence resistance to folate analogs without adversely affecting TS enzyme activity.

Best results were obtained with the following mutants: D254N-D254A (double), D254E (single), T53S (single), Y258F (single), T53S-Y258F (double), T51S-K82Q-K99E-N171S (quadruple), G5D-L31R-A231 T (triple), D254A (single), D254N (single). The results are given in greater detail in Tables I and II.

TABLE I
Mutation spectra produced by amplification of human TA cDNA^{a}

| | | Average | | Number of Substitutions | | | | | | |
|---|---|---|---|---|---|---|---|---|---|---|
| | | No. of | Mutations | Transitions | | Transversions | | | | |
| PCR | No. of | mutations | frequency | AT [symbol] | GC [symbol] | AT [symbol] | AT [symbol] | GC [symbol] | GC [symbol] | |
| Conditions | clones | per clone | (%) | GC | AT | TA | CG | TA | CG | Deletions |
| Standard | 36 | 0.2 | 0.031 | 6 | 0 | 0 | 0 | 0 | 1 | 0 |
| Mutagenic | 18 | 9.8 | 1.58 | 41 | 35 | 66 | 3 | 3 | 0 | 7 |

^{a}The cDNA encoding human TS was PCR-amplified under standard and mutagenic conditions, cloned into pCRII-TOPO, and the 5′-terminal two thirds of the DNA (nucleotides 1-630) was sequenced.

a The cDNA encoding human TS was PCR-amplified under standard and mutagenic conditions, cloned into pCRII-TOPO, and the 5′-terminal two thirds of the DNA (nucleotides 1-630) was sequenced.

TABLE II
5-FdUR-resistant human TS mutants created by error-prone PCR^{a,b}

| Substitution(s) | | |
|---|---|---|
| Survival^{C} | Mutant Number | Mutant/WT |
| Wild type none | — | |
| E32G | 357 (a) | >110 |
| D48V | 312 (a) | >98 |
| V84A | 319 (b) | 54 |
| K93E | 209 (c) | 340 |
| D110E | 330 (b) | 530 |
| P194Q | 226 (d) | 36 |
| S206G | 229 (e) | >24 |
| H250L | 339 (a) | >7 |
| K284N | 331 (a) | >14 |
| P121L, N302S | 307 (f) | >37 |
| D21G, M311L | 217 (d) | 56 |
| V45A, D254N | 255 (e) | >210 |
| T53S, Y258F | 302 (g) | 220 |
| G54C, M236I | 329 (f) | >180 |
| F80S, F91L | 219 (h) | >90 |
| F80S, L88S | 353 (f) | >250 |
| L85M, L88S | 361 (i) | >62 |
| T96S, A228T | 370 (e) | >300 |
| S103T, V204A | 316 (f) | >140 |
| F142S, F225I | 317 (f) | >200 |
| P172S, D254E | 358 (e) | >180 |
| P193S, A231G | 308 (g) | 270 |
| G5D, L13R, A231T | 315 (g) | 280 |
| G5S, R78C, M219I | 228 (e) | >82 |
| S6N, D69Q, Q211L | 313 (f) | >140 |
| A17T, D116A, D254E | 318 (b) | 770 |
| Q18R, R78H, I262T | 238 (i) | 54 |
| T55I, V106A, K284I | 222 (h) | >48 |
| S66G, K104R., E128G | 369 (f) | >210 |
| L8Q, D21G, t53A, R78L | 203 (e) | >320 |
| L8Q, W81G, L131V, Y230F | 354 (f) | >170 |
| L13Q, P27L, R42H, V84A, | 356 (f) | >190 |
| F248L | | |
| V45I, T125I, A144S, K308R | 326 (b) | 16 |
| T51S, K82Q, K99D, N171S | 362 (g) | 180 |
| T170S, V204M, S206G, | 359 (f) | >160 |
| M219V | | |

The resistance of each of the 41 mutants to 5-FdUR is expressed as fold enhancement in survival relative to wild type determined in parallel assays. All assays were carried out in duplicate at varying concentration of 5-FdUR and most comparison were repeated in two or more separate experiments. As previously reported, cells expressing wild type TS exhibited a diminished ability to form colonies on M9 plates containing more than 150 nM 5-FdUR. In most but not all experiments no wild type colonies were observed at concentrations of 150 nM 5-FdUR or greater. Only those experiments in which the survival of the wild type was lower than 1% were included in this tabulation. The percent survival of the wild type at the indicated concentrations of 5-FdUR is as follows: (a) 0.4% in 0.4 nM; (b) <0.1% in a 150 nM; (c) 0.1% at 150 nM; (d) 0 at 300 nM; (e) 0.5% at 150 nM; (f) 0 at 200 nM; (g) 0.95 at 150 nM.

The invention also encompasses recombinant cDNAs encoding for the above given mutants as well as vectors comprising these DNA sequences and—optionally—other components.

The invention also encompasses fusion proteins comprising a transductor for the transduction of a protein into a human cell and a human thymidylate synthase mutant of the invention coupled to said transductor. Methods for the production of fusion proteins can be taken from “Schwarze, et al., Science 285:1569, 1999”. Transduction can also be achieved with the help of viral vectors, liposomes or complexing agents. The fusion proteins where an amino acid sequence attached to the TS mutant is used for protein transduction are well suited for the introduction of the mutants of the invention into mucosal cells, especially oral mucosal cells, in culture or in vivo.

The TS mutants according to this invention can be expressed in either bacteria or yeast as host cells.

The protein mutants, respective DNAs, vectors or fusion proteins according to this invention can be used for transfection of human cells which may be affected by side effects due to chemotherapy with thymidylate synthase inhibitors, for the ex-vivo or in vivo transfection of bone marrow cells, or for transfection of early progenitor cells separated or grown from bone marrow cells. Vectors and DNAs can be used in gene therapy, where they are introduced into individuals harboring tumors. This is especially intended for protection of human mucosa against ulceration in patients undergoing chemotherapy with agents that inhibit TS. The means of this invention are also useful for introduction of TS mutants into normal (oral or other) mucosa in patients prior to chemotherapy.

The protein mutants of this invention, the respective DNAs, vectors or fusion proteins can also be used for the identification of new nucleotide analogs that inhibit normal human thymidylate synthase.

The protein mutants, respective DNAs, vectors or fusion proteins according to this invention are useful for the manufacture of pharmaceutical and gene therapeutic compositions for the protection of human mucosa against ulceration under chemotherapy with TS inhibitors. The invention encompasses such pharmaceutical or gene therapeutical compositions.

The inventors used error prone PCR to mutagenize the full length human TS cDNA, and then selected mutants resistant to 5-fluorodeoxyuridine (5FdUR) in a bacterial complementation system. They found that resistant mutants contained one to five amino acid substitutions, and that these substitutions were located along the entire length of the polypeptide. Mutations were frequent near the active site Cys195 and in the catalytically important Arg50 loop; however, many mutations were also distributed throughout the remainder of the cDNA. Mutants containing a single amino acid replacement identified the following 14 residues as novel sites of resistance: Glu23, Asp48, Thr51, Thr53, Val84, Lys93, Asp110, Asp116, Met 190, Pro194, Ser206, Met219, His250, Asp254, Tyr258, and Lys284. Many of these residues are distant from the active site and/or have no documented function in catalysis or resistance. It was concluded that mutations distributed throughout the linear sequence and 3dimensional structure of human TS can confer resistance to 5FdUR. The findings imply that long range interactions within proteins affect catalysis at the active site and that mutations at a distance can yield variant proteins with desired properties.

Amongst the TS variants containing a single amino acid substitution, the inventors identified replacements at residues that have not been previously reported to be sites of resistance in human TS. In fact, this more than doubles the number of single amino acid substitutions in human TS that have been demonstrated to yield 5FdUR resistance.

Another object of the present invention is to provide a drug for the treatment or prevention of a chemotherapy-induced mucositis and other side effects associated with a chemotherapy available, which has a causal effect instead of being symptomatically effective. The drug shall be specifically locally applicable in order to be able to administer it directly to the affected tissues.

In order to solve this problem it is intended, to design a drug for the local treatment or prevention of a mucositis or other side effects in the gastrointestinal tract including the oral cavity, which are caused by chemotherapy with thymidylate synthase inhibitors. This drug will contain at least one thymidylate synthase mutant either in form of a protein or a nucleic acid. The thymidylate synthase mutant will be linked to a transducer, which causes the intake of the drug into the cells that need to be treated. The thymidylate synthase mutant reveals a completely intact enzymatic activity and is resistant to thymidylate synthase inhibitors.

Contrary to the local application of merely symptomatically effective drugs, which are frequently ineffective for the treatment of a chemotherapy-associated mucositis, according to the present invention, active TS-mutants are used which are resistant to TS inhibitors and thus are causally effective against the cell destruction, which is causative for a mucositis.

An important aspect of this invention is, to infiltrate the TS mutants as easy, effective, and selective as possible into the target cells. Due to obvious reasons it must be avoided, to infiltrate TS mutants into tumor cells too, since this would interfere with the essential therapy, the chemotherapy with TS inhibitors. Therefore it is intended, to couple the TS mutants with a “transporter molecule”, the so-called transducer, which can mediate the transport, i.e. the transduction, into the target cell. The transduction shall be preferentially locally, i.e. specifically into the mucosa, which is affected by the chemotherapy, but under no circumstances systematically. For this, various possibilities are available.

Generally, the transduction of the protein or the gene can be performed with any appropriate method, which is known to the expert. Many transduction techniques have been described in the literature. This includes the use of cationic liposomes, viral vectors or the coupling with various transduction proteins or peptides in general.

A further possibility for the protein transduction is, to couple the TS mutants with a transporter protein, which can penetrate the biological cell membrane. During the past decade various transduction proteins (PTDs, Protein Transduction Domains) were found. This includes among others the TAT protein of HTV, the drosophila homeotic transcription factor (ANTP) and the herpes simplex virus type I (HSV-1) VP 22 transcription factor. Appropriate PTDs and their in vivo application have been already described (Schwarze and Dowdy, 2000). It was possible, to deliver active enzymes and DNA into all tissues by means of PTDs, even passing the blood-brain-barrier. It could be demonstrated with more than 60 TAT-fusion proteins that a transport (a transduction) was successful in nearly 100% of the investigated cell types (primary and immortalized cells) as well in cells/tissues of mice within 5 minutes.

The transport of TAT fusion proteins in mammalian cells is described in laboratory manuals (Dowdy S F. 2000), TAT-derived transport polypeptides have been already disclosed (Barsoum et at., 1992). These can deliver polypeptides and nucleic acids in the cytoplasm and the nucleus of cells in vitro and in vivo. The intracellular delivery of freight molecules is achieved by means of transport polypeptides, which comprise one or several parts of the HIV-TAT-protein and are covalently bound to the freight molecules.

According to the present invention, the drug is designed for the local application in the gastrointestinal tract including the oral cavity. A preferred type of preparation is an oral rinsing solution. Another preferred type of preparation are protease protected and acid resistant capsules, coated tablets (dragees), or granulates that contain the TS mutants and which are produced in a way that is familiar to the expert. These forms of administration allow the passage of the stomach without destruction of the transducer coupled TS mutants so that they can arrive intact in the intestine. This way the mucosa or related tissues of the intestine can be also protected by transduction of IS mutants against unwanted side effects of chemotherapeutic drugs, which contain TS inhibitors.

Another preferred type of administration of TS mutants for the therapy of the mucosa or related tissues of the intestine are rectally applied types of administration, such as clysmas (enemas) or suppositories, which allow the bypassing of the acidic gastric environment. The treatment of the gastric mucosa requires the pretreatment with oral acid blockers, which are used in medicine since many years for a short-term neutralization of the acidic environment. Acid sensitive drugs such as the transducer coupled TS mutants can be also administered to the mucosa of the stomach by means of an orally applied therapeutic subsequently to such a pretreatment.

Another preferred type of administration would be the application of viable bacteria in the intestinal micro flora of the patient under chemotherapy. These bacteria should be modified in such a way that the express the appropriate TAT-TS fusion proteins. The microbial strain, which is used for such a therapeutic strategy, should be selected in such a way that it can survive and proliferate in the physiological intestinal flora for at least several days. But on the other hand, a certain share of these bacteria should be also lyzed in the intestinal environment in order to liberate the TAT-TS-fusion proteins which are expressed by this microbial strain. Alternatively, other microorganisms, such as yeast, could be used, which are capable of secreting expressed proteins. In principle, all types of organisms are appropriate, which can survive in the human intestinal flora and which are compatible with human health. The bacteria could be applied either orally by means of acid-resistant capsules or rectally by means of suppositories.

Dependent of the type of administration various adjuvants, additives and fillers for the production of drugs, which contain transducer-coupled TS mutants can be applied. Oral rinsing solutions and clysmas (enemas) can be formulated as physiological saline solution. The pH of this solution can be adjusted to physiologic values for instance by means of potassium hydrogen phosphate and potassium-di-hydrogen phosphate. In order to secure the stability of the TS-mutants, accessory substances such as reducing agents or mild detergents, which are compatible with proteins, can be used.

Proteins, such as casein or human albumin, can be added to the drug for additional stabilization of the TS mutants. If necessary, preparations containing the transducer coupled TS mutants, which will be used as liquids, can be made available as dried powders and are dissolved in an appropriate diluent, such as water, immediately before application.

Other substances known to the skilled person, such as methacrylic acid polymer, macrogol, carnauba wax and other waxes, shellac, polyvidone, cellulose, talcum, calcium stearate, xanthane rubber, hard fats, silicon oxide and other appropriate adjuvants, additives or filler can be used. For instance, sorbic acid or sodium benzoate can be added as preservatives. Additional adjuvants or additives, which are known to the skilled person, can be added such as coloring, flavorring substances or fragrances.

The cells of the gastrointestinal tract (mucosa cells), which are affected by side effects due to a chemotherapy with TS inhibitors, can be locally reached particularly fast, efficient and selective. According to a presently preferred embodiment invention, protein mutants with a TAT transporter protein are infiltrated into the cells. According to the invention, this system is particularly appropriate, since animal experiments revealed that TAT fusion proteins can be transduced into primary cells within 5 minutes. Thus, TAT fusion proteins are specifically suitable for a topical application, for instance as oral rinsing solution. Transducers, which need a long period of time to fulfill their function, are generally less appropriate for a use in rinsing solutions. In another preferred embodiment Herpes VP22 fusion proteins can be used for the transduction of TS mutants instead of TAT fusion proteins. Further, transport proteins, which are optimized for transduction by means of site directed or random mutagenesis, can be applied.

Further, the non-covalent complex formation according to the Chariot™ protein transfection technique can be used in the sense of this invention (2001. Chariot: the vehicle of the future for protein transfection. Active Motif, Carlsbad, Calif., USA, 15 pp). The complexing substance allows the penetration of the biological cell membrane in a short period of time. Simultaneously, the transported protein is protected in the non-covalent complex. Due to the short transduction times up to 2 hours complex substances are specifically useful as transducers for the direct, local application of relatively short-lived protein mutants (life span minutes up to 24 hours). Other complexing substances, which are useful for the aforementioned purpose, are also applicable.

The invention includes the direct use of TS mutants as isolated proteins on the one hand, and their application as nucleic acids, which encode these TS mutants and thus make the expression of the TS mutants in the target cells possible on the other hand. Potential nucleic acids for example would be mRNA of IS mutants or expression vectors TS mutants with constitutively active and inducible promoters. Since there is only a transient expression of the TS mutants necessary for the treatment of chemotherapy-associated side effects, only those systems should be used in case of expressions vectors, which are only active for a few days or weeks and which are not incorporated into the genome of the patient. In order to avoid this problem TS mutants could be preferentially transduced as proteins or mRNA. The infiltrated or in the cells generated IS mutants display their physiological functions in the (mucosa) tissue preventively or as therapeutic of already damaged cells. Their special feature is that they are not or only little inhibited by the TS inhibitors, which are used for the chemotherapy. TS mutants as proteins as well as TS mutant coding nucleic acids can be coupled to the same transducers (complexing substance/PTDs). TS nucleic acids, which encode TS inhibitor resistant proteins, are preferentially transduced into the different types of cells of the mucosa or other target tissues by means of transport proteins (PTDs), which can be optimized for the specific genes concerning the transduction efficiency on the other hand.

The TS nucleic acid mutants will be produced by random or site directed mutagenesis and checked for the desired characteristics. The accompanying protein should reveal a good stability combined with a high enzymatic activity and resistance to TS inhibitors. The generation and characterization of TS mutants is comprehensively described for example in WO 98/33518, which is referred to regarding the experimental data of mutants

The resistance of the single mutants can be tested with different TS inhibitors. Presently, 5-FU (5-fluorouracil), which is metabolized to FdUMP (fluorodeoxyuridylate), FdUMP or folate analoga, such as Raltitrexed™, Thymitaq™ or AG331 are mainly applied. Therefore, the mutants will be tested for resistance to these substances. Additional Ts inhibitors can be included in necessary.

The TS mutants are preferentially generated by means of cloning cDNA of resistant TS mutants in bacterial expression vectors (Dowdy, 2000).

As further stage of this invention it is also intended that the transduction efficiency of the transport proteins, which are used in this invention, will be optimized. The transductions efficiency arises from the speed of penetration of the fusion protein, which consists of the particular PTD and the TS mutant and the portion of molecules which successfully infiltrated the cells. This can be achieved by random or site directed mutagenesis of the particular PTD and subsequent analysis of the transduction characteristics.

The success of the proposed application will be significantly improved by optimizing the transport protein, i.e. the transducer, towards the best possible transduction efficiency for the particular TS mutant. It is obvious that the success of a treatment of a mucositis is essentially dependent on infiltrating the target cells with the highest possible amount of nucleic acid or protein mutants as fast as possible and maintaining the function or activity of the mutant. It is essential for this goal to optimize the PTD.

It is referred to the information and data of all papers, which are cited in this application.

The invention will be demonstrated using the following examples.

### EXAMPLES

### Example 1

### Resistant Thymidylate Synthase Mutants for Locally Treating a Chemotherapy-Induced Mucositis and Other Side Effects Associated with Chemotherapy

Experimental Procedures

Abbreviations

The abbreviations used are: CH₂H₄-folate, (6R,S)-N⁵,N¹⁰-methylene-5,6,7,8-tetrahydrofolate; 5-FdUR, 5-fluoro-2′-deoxyuridine; FdUMP, 5-fluoro-2′-deoxyuridine 5′-monophosphate; 5-FU, 5-fluorouracil; bp, base pair(s); PCR, polymerase chain reaction; PAGE, polyacrylamide gel electrophoresis; TES, N-tris[hydroxymethyl]methyl-2-aminoethane-sulfonic acid; TS, thymidylate synthase.

The human numbering system of TS is used for consistency. H256 corresponds to H207 in E. coli, Asp254 is Asp209 in E. coli, Arg175′ is Arg126′ in E. coli, and Tyr258 is Tyr 261 in E. coli. The prime indicates residues contributed by the opposing homodimer.

Cell Lines and Materials

E. coli NM522 cells (Stratagene, La Jolla, Calif.) were used for cloning and library construction. E. coli c2913recA cells lacking TS (ΔthyA572, recA56), kindly provided by Dr. Daniel Santi (UCSF), were used in all complementation studies. Plasmid DNA was isolated by using Perfectprep Plasmid Mini (Eppendorf Scientific Inc., Westbury, N.Y.) and Maxiprep kits (Qiagen, Charsworth, Calif.). Iaq DNA polymerase and dNTPs were from Promega (Madison, Wis.). DNA oligomers and T4 DNA ligase were from Gibco Life Science Technology. Restriction enzymes were obtained from New England Biolab (Beverly, Mass.). DNA fragments were isolated by using the Quiaquick Gel Extraction Kit from Qiagen. 5-FdUR, FdUMP, thymidine were purchased from Sigma (St. Louis, Mo.), and (6R,S)-CH₂H₄-folate was obtained as the racemic mixture from Schircks Labs (Jona, Switzerland). ABI Prism Dye Terminator Cycle Sequencing kits for fluorescent sequencing were the products of Perkin Elmer (Branchburg, N.J.).

Standard and Error-Prone PCR

Standard PCR reactions contained 100 ng plasmid DNA carrying the wild type TS cDNA, 1 μM of the primers HK-MUT5′ (5′-ATAACAATTTCACACAGGAAACAGCTATGACC-3′, SEQ ID NO: 1) and HK-MUT3′ (5-′ CAGGGTTTTCCCAGTCACGACGTTGTAAAACG-3′, SEQ ID NO: 2), 250 μM each dCTP, dTTP, dATP and dGTP, and 2 units of Taq DNA polymerase in 10 mM Tris-HCl (pH 8.3) 1.5 mM MgCl₂, 50 mM KCl, 0.01% gelatin, 0.01% Triton X-100 in a total volume of 100 μl. Error-prone PCR was performed by the method of Cadwell and Joyce (17) with modification. Conditions were those described for standard PCR, except for elevation of the MgCl₂ concentration to 7 mM, addition of 0.5 mM MnCl₂ and unequal concentrations of the four dNTPs (1 mM dCTP, 1 mM dTTP, 0.2 mM dATP,0.1 mM dGTP). PCR was performed in a PCT-100 Programmable Thermal Controller (MJ Research Inc., Watertown, Mass.) by using the following protocol: 3 min of initial denaturation at 94° C. followed by 45 cycles of 94° C. for 1 min, 50° C. for 1 min and 72° C. for 3 min. To determine the mutation frequency under standard and mutagenic conditions, PCR products were cloned into the pCRII-TOPO vector (Invitrogen, Carlsbad, Calif.) and sequenced by using the primer, DLPCR-TS3R (5′-AAAAAAAAACCATGTCTCCGGATCTCTGGTAC-3′, SEQ ID NO: 3).

Construction of the TS Mutant Library

DNA amplified by error-prone PCR was digested with Sad and NdeI and subjected to electrophoreses in an agarose gel. The 1 kb TS fragment was extracted from the gel by using the Qiaquick Gel Extraction kit (Qiagen) and ligated into the Nde1 and Sac1-digested pGCHTS-TAA vector backbone (kindly provided from Dr. Daniel Santi) containing 115 bp of 5′-untranslated region from the L. casei TS gene as a leader sequence. The ligated plasmid DNA was transformed into NM522 E. coli cells (Stratagene) by electroporation (Gene Pulser, BioRad, 1.8 kV, 25 μF, 400 ohms). Twenty five transformations were pooled and the size of the library was determined to be 2.8×10⁶ independent clones by plating an aliquot of the transformation mixture on 2×YT medium containing 50 μg/ml of carbenicillin (Island Scientific, Bainbridge Island, Wash.). To determine the frequency and types of mutations introduced by PCR, plasmid DNA was isolated from the E. coli mutant library and sequenced by using the ABI Prism Dye Terminator Cycle Sequencing Kit and the primer DLPCR-TS3R. For amplification of the library, 25 ml of the pooled transformation mixture was added into 500 ml of 1×YT medium containing 50 μg/ml of carbenicillin, incubated overnight at 37° C., and plasmid DNA was prepared. The plasmid DNA library (500 ng) was transformed into 100 μl of electrocompetent χ2913 cells. Transformation mixtures were pooled, grown overnight in appropriate antibiotics and stored at −80° C. in 15% glycerol.

Genetic Selection in E. coli

E. coli χ2913 cells harboring plasmids with random substitutions encoding different amino acids, the TS library, were grown overnight at 37° C. in 2×YT medium containing 50 mg/ml of carbenicillin and 50 mg/ml thymidine. Cultures were diluted 1:100 in the same medium and grown at 37° C. until the OD₆₀₀ reached 0.6 to 0.8. Aliquots (1 ml) of the exponentially growing cells were pelleted and resuspended in M9 salts and plated on three different media. On 1×YT containing 50 mg/ml of carbenicillin and supplemented with 50 mg/ml of thymidine, essentially all cells harboring the plasmid DNA should be recovered. On M9 plates containing carbenicillin, only cells expressing active TS proteins should grow because the medium lacks thymidine. Drug resistant clones were selected on M9 plates containing carbenicillin and increasing concentrations of 5FdUR essentially as previously described (Landis, D. M., Loeb, L. A., 1998, J. Biol. Chem. 273:25809-25817)

Site-Directed Mutagenesis

Single amino acid substitutions were introduced by using the QuikChange site-directed mutagenesis kit (Stratagene). Plasmid DNA encoding the wild type TS cDNA was amplified by using PfuTurbo DNA polymerase and a set of primers containing the appropriate base substitution. The PCR product was treated with DpnI to cleave the parental DNA template and the noncleaved DNA was transformed into XL-1 Blue competent cells. Cells containing plasmid DNA with the desired substitution were selected on the LB ampiciilin agar plates, and plasmid DNA was harvested and sequenced.

Purification of TS Proteins

To subclone mutant TS fragments into the pH is vector (a modified pUC12 vector provided by A. Hizi), the TS cDNA was PCR-amplified by using HK-MUT5′ and TSXho3′ (5′-CAGCTCGAGCTCCTTTGAAAGCACCCTAAAC-3′, SEQ ID NO: 4). After digestion with NdeI and XhoI, the amplified fragments were ligated into the NdeISalI digested pH is vector. The reconstructed plasmids were verified by both restriction analysis and DNA sequencing. Wild type and mutant TS proteins, as N-terminal hexahistidine fusions, were then purified by metal chelation chromatography on Ni²⁺ affinity resin (His Bind resin and buffer kit, Novagen), as previously described, with minor modifications (see Landis, cited above). An overnight culture of χ2913 cells expressing the His-TS fusion protein was diluted 1:100 into 250 ml of 2×YT medium containing carbenicillin and thymidine. After attaining an OD₆₀₀ of 0.2, cells were induced with 1 mM IPTG, grown to an OD₆₀₀ of 0.8, harvested by centrifugation, resuspended in 12 ml of 1× binding buffer (5 μM imidazole, 0.5 mM NaCl, 20 mM Tris-HCl, pH 7.9) and lysozyme (200 μg/ml, Sigma), and frozen at −80° C. Frozen cells were thawed and lysed on ice for 3 hr. The disrupted cells were centrifuged (27,000×g) and the supernatant was collected and applied to a charged 2.5 ml HisBind Column (1×2.5 cm, Novagen, Madison, Wis.). The resin was prepared by successive washes with 15 ml of deionized water, 10 ml of 1× charge buffer (50 mM NiSO₄), and 15 ml of 1× binding buffer. Following application of the crude extract, the column was washed with 30 ml of 1× binding buffer and 25 ml of a mixture containing 60% binding buffer and 40% wash buffer (60 mM imidazole, 500 mM NaCl, 20 mM Tris-HCl, pH 7.9). His-TS protein was eluted with 5×1 ml aliquots of 1× elution buffer (1 M imidazole, 0.5 mM NaCl, 20 mM Tris-HCl, pH 7.9). SDS-PAGE showed that TS protein was eluted in the second and third fractions. Fractions containing TS protein were combined and dialyzed against 50 mM Tris-HCl, pH 7.5, 1 mM EDTA, 200 mM NaCl and 10% glycerol for 16 h and then against the same buffer containing 1 mM dithiothreitol for additional 10 h. The concentration of purified TS was determined by using the Bradford assay.

Kinetic Analysis

TS activity was monitored spectrophotometrically by the increase in absorbance at 340 nm that occurs concomitant with the production of H₂ folate (Δε=6400 M⁻¹ cm⁻¹). The reaction buffer contained 50 mM TES (pH 7.4), 25 mM NaCl, 6.5 mM formaldehyde, 1 mM EDTA and 150 μM 2-mercaptoethanol. When the concentration of dUMP was varied, a high concentration of (6R)—CH₂H₄ folate was added (3001200 mM of a racemic mixture); when the concentration of (6R)—CH₂H₄-folate was varied, the concentration of dUMP was 400 μM. Purified wild type or mutant TS protein was added to initiate the reaction at 25° C. Initial velocity measurements were obtained with a Perkin Elmer Lambda Bio 20 UV/VIS spectrophotometer. Steady-state kinetic parameters were derived by a nonlinear least squares fit of the data to the Michaelis Menten equation with KaleidaGraph 3.0 software (Abelbeck Software, Reading Pa.). Inhibition constants (Ki) for 5-FdUMP were obtained from a Dixon plot by measuring initial velocities in the presence of dUMP, CH₂H₄-folate and inhibitor at 25° C. Two dUMP concentrations (30 μM and 300 μM) were used, and FdUMP concentrations were varied. To initiate reactions, CH₂H₄-folate was added to yield a final concentration of 300 μM of the active isomer.

Results

Mutagenesis of Human TS by Error-Prone PCR

To introduce amino acid substitutions throughout the TS protein, error-prone PCR was carried out by increasing the concentration of Mg²⁺, adding Mn²⁺, and using unequal concentrations of the four dNTPs, Manganese ions have been shown to increase misincorporation by a variety of DNA polymerases, and the error-rates of these enzymes are proportional, over a limited range, to the relative concentrations of the dNTP substrates. The mutations introduced by PCR under standard and mutagenic conditions were compared by sequencing the initial two-thirds of the TS cDNA (nucleotides 1-630 encoding amino acids 1-210.) Under standard conditions with 1.5 mM Mg²⁺ and equimolar dNTPs, Taq DNA polymerase introduced a relatively small number of mutations; the average for the 36 clones sequenced was 0.2 mutations per clone. Under mutagenic conditions, however, the average was much higher, i.e., 9.8 mutations per clone, and the great majority of cDNAs carried more than 7 mutations (Table I). Six of 7 mutations found under standard conditions were A:T to G:C transitions, frequently introduced by Taq DNA polymerase. In contrast, under mutagenic conditions, A:T to T:A transversions were most frequent, and a variety of other types of substitutions were detected, predicting a greater diversity of amino acid substitutions.

Construction of TS Mutant Library and Analysis of Unselected Clones

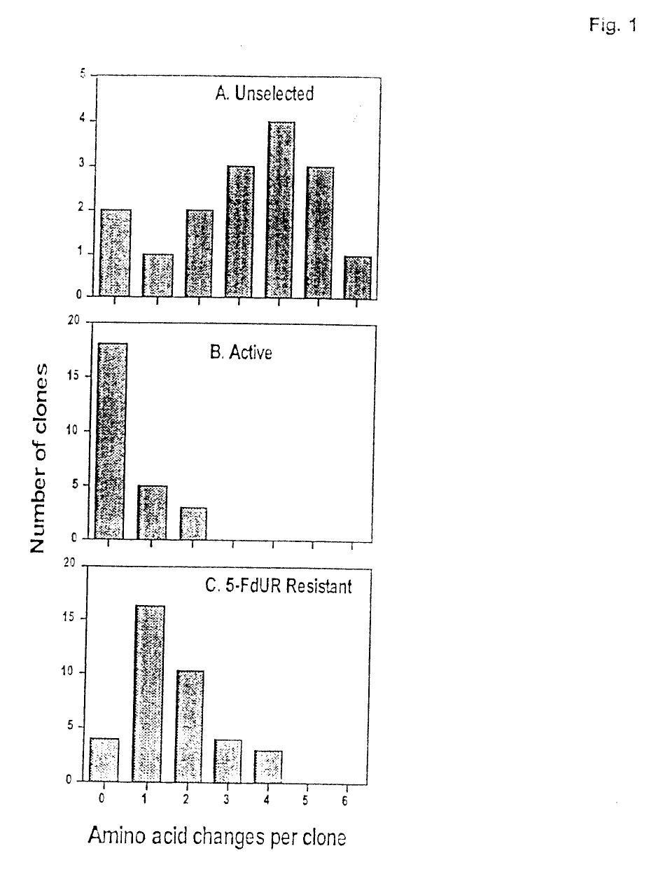

Following amplification by error-prone PCR, the TS cDNAs were cloned into a plasmid vector downstream of the L. casei TS untranslated sequence. The ligated products of the backbone and these variant PCR products constituted the TS mutant library, containing ca. 2.8×10⁶ independent clones. Plasmid DNA was isolated from 16 colonies obtained from growth on non-selective rich medium containing thymidine, and the number and types of nucleotide substitutions were determined by sequencing the 5′-proximal 630 nucleotides of the cDNA. The number of mutations per cDNA in the non-selected mutants is shown in FIG. 1A. Unselected clones have an average of 4.3 nucleotide changes, all of those detected were base substitutions with the exception of 2 deletions. At the amino acid level, this corresponds to 3.2 substitutions within the segment sequenced, suggesting that, on average, the full-length proteins contained about 4.8 amino acid replacements. Amino acid substitutions in the unselected clones were distributed fairly uniformly throughout the sequenced region (data not shown).

Selection and Analysis of Active TS Mutants

Expression of catalytically active TS protein enables TS-deficient E. coli χ2913 host cells to form colonies on M9 minimal plates in the absence of added thymidine. On plating the TS mutant library, approximately half the number of colonies was formed on M9 minimal medium lacking thymidine relative to the thymidine-supplemented medium. Thus, approximately 50% of the TS mutants exhibited sufficient catalytic activity to complement the TS-deficient phenotype. Of the active mutants analyzed, 8 of 18 clones carried amino acid substitutions in the 630 bp sequenced (FIG. 1B). No amino acid substitutions were detected in the other 10 clones, although two clones contained silent mutations. This limited survey indicates that most of the active mutant clones contained less than one amino acid substitution and that these substitutions do not prevent the catalytic activity required for complementation.

Isolation and Analysis of 5-FdUR-Resistant TS Mutants

To isolate mutants that render E. coli χ2913 resistant to 5-FdUR, the TS library was plated on M9 minimal medium containing 200 or 300 nM 5-FdUR. As previously reported, cells expressing wild type TS exhibited a diminished ability to form colonies on M9 plates containing more than 150 nM 5-FdUR. Resistant colonies were selected and plasmid DNA was isolated and re-transformed into fresh χ2913 cells to confirm that the plasmid was the source of the drug-resistant phenotype. Thirty-six independent clones were picked that showed increased resistance upon re-exposure to 5-FdUR, and the 5′-terminal 630 bp of the cDNA was sequenced. Zero to 4 amino acid substitutions were observed in the sequenced segment of resistant clones, the average being 1.6 (FIG. 1C).

To obtain a profile of amino acid replacements conferring resistance, the remaining, 3′-proximal third of the cDNA from each of the 33 mutants was sequenced. In two of the clones we failed to detect any substitutions; the mechanisms of resistance in these two clones remains to be determined. A summary of all predicted amino acid substitutions found in 36 of the mutants that exhibited resistance to ≧150 nM 6-FdUR is recorded in Table II. Resistance is recorded as percent survival relative to percent survival of host cells harboring wild type TS at 150 nM 5-FdUR of greater. Expression of TS in 10 of the resistant mutants was analyzed by Western blotting with antiserum directed against human TS. The level of expression of wild type TS was more than 2-fold greater than that of any of the mutants, estimated by visual inspection of the gel (data not shown). The mutant proteins may be more labile than the wild type or more susceptible to proteolytic degradation.

Distribution of PCR-Generated Mutations Yielding 5-FdUR Resistance

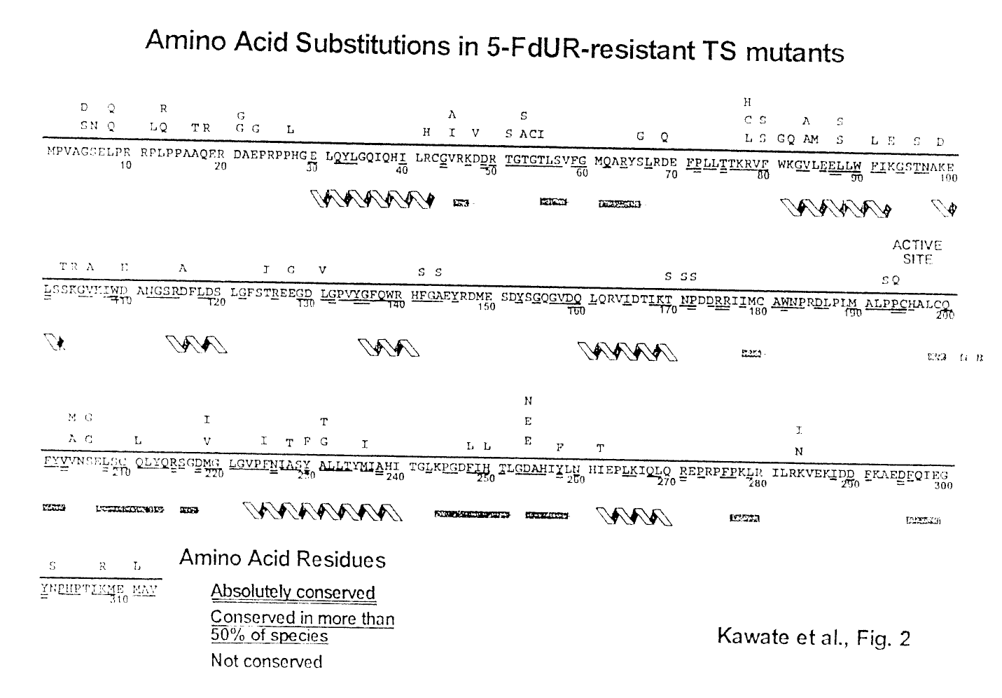

The distribution of amino acid substitutions in the 5-FdUR-resistant clones generated by error-prone PCR is indicated in FIG. 2. In the case of mutants with multiple substitutions, not all of the replacements are expected to be directly involved in resistance, some presumably having been co-selected. Importantly, substitutions were dispersed throughout the protein. The distribution included “hot spots” where clustered mutations appeared to be more frequent than would be expected on a random basis, as well as “cold spots.” Several regions exhibited a high density of mutations. The residues between and including Val45 and Thr55 constituting the Arg50-loop harbored 7 different substitutions. The replacements included T51S, commonly observed, and present in most resistant mutants, in previous experiments. These results are in accord with previous reports showing that the evolutionarily conserved Arg50-loop is a major site of mutations that confer 5-FdUR resistance.

The residues from Arg78 to Leu88 accumulated 9 replacements. This region has not been reported to be involved in 5-FdUR resistance; structural analysis indicates that it is located on the active site pocket and interacts with folate-based inhibitors. The N-terminal residues from Gly5 through Gln18 accumulated 10 substitutions; this segment is disordered in existing crystal structures and has no known function. There is no obvious correlation between the frequency of either single or multiple substitutions observed in resistant mutants and the presence of beta sheets or alpha helices. However, amino acid residues that are completely conserved during evolution are less likely to incur substitutions resulting in 5-FdUR resistance.

Resistance of T53S;Y258F Reflects the Contribution of Both Single Mutations

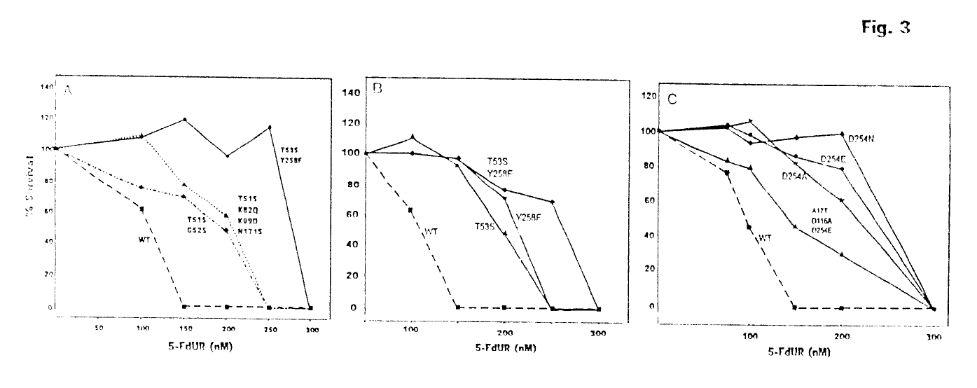

Amongst the newly identified clones, the double mutant T53S;Y258F (clone 302), exhibited a high level of resistance at elevated concentrations of 5-FdUR. In multiple experiments, T53S;Y258F was as resistant or more resistant to elevated concentrations of 5-FdUR than any clone tested. As shown in FIG. 3A, E. coli cells carrying the T53S;Y258F mutant grew on minimal plates containing 250 nM 5-FdUR, whereas, in a side-by-side comparison, the double mutant T51S;G52S, formed colonies at 200 nM, but not 250 nM, 5-FdUR. The same was true for another clone that exhibited high-level resistance, the quadruple mutant T51S;K82Q;K99E;N171S (clone 362), as well as for another triple mutant G5D;L31R;A231T (clone 315, results not shown). To analyze the contribution of individual amino acid substitutions to the 5-FdUR resistance of the double T53S;Y258F mutant, we generated the T53S and Y258F single mutants by site-directed mutagenesis. As illustrated in FIG. 3B, both the T53S and Y253F single mutants were less resistant than the double mutant, but more resistant than wild type TS. These results indicate that both substitutions contribute to the high level of resistance exhibited by T53S;Y258F.

Asp254 is a Site of 5-FdUR Resistance

Replacements at Asp254 were detected in 3 multiply substituted, resistant clones generated by error-prone PCR (D254E in 2 clones, and D254N in one). We generated several single mutants at this site and assessed their resistance. As shown in FIG. 3C, the three mutants D254A, D254E and D254N all showed higher resistance than wild type TS and were more resistant than a parent clone (clone 318) which had two substitutions in addition to D254E (A17T;D116A; D254E).

Kinetic Analysis of Mutant TS Proteins

To establish that the resistance to 5-FdUR observed in E. coli reflects altered function of the mutant proteins, several TS variants were purified as N-terminal hexahistidine fusions. Purity was estimated to be greater than 80% by densitometric scanning of Coomassie Blue-stained gels. Steady-state kinetic parameters for the wild type and mutant TS proteins are recorded in Table III. The kcat values observed for the mutants, and the Km values for dUMP, do not differ markedly from that of wild type. However, the Km values of the mutants for CH₂H₄-folate were 5- to 13-fold higher than that for wild type TS. Km's were approximately 10-fold higher for the double mutant 153S; Y258F (clone 302) and the quadruple mutant T51S; K82Q; K99D; N171S (clone 362), which were among the most resistant variants. In accord with the E. coli survival data, the Ki of all mutants for 5-FdUMP was greater than that of wild type TS. Mutant T53S; Y258F (clone 302), which exhibited high resistance to 5-FdUR in our complementation assay, showed a 6-fold increase. T51S demonstrated the largest increase over wild type (11-fold), while the less resistant single mutant, Y258F (see FIG. 3B), showed a lesser increase (3.1 fold).

Single Mutants that Yield 5-FdUR Resistance

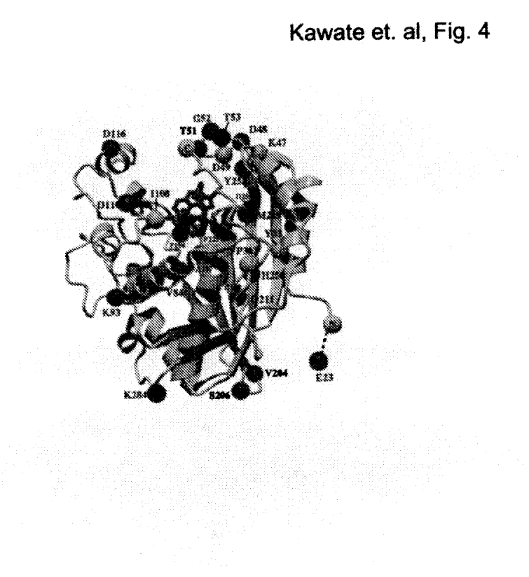

The hypothesis we set out to assess is that mutations in TS that confer 5-FdUR resistance are distributed throughout the protein, and are not confined to regions directly involved in catalysis. In this work, we created single mutants that newly identify a total of 14 amino acids as residues where 5-FdUR resistance can arise. Eight residues were identified in mutants created by error-prone PCR; these are E23, V84, K93, D110, P194, S206, H250, K284. Six of these (E23,V84, K93, D110, S206, K284) are located at a distance from the active site and are not associated with catalysis. We also identified other single amino acid substitutions that may confer 5-FdUR resistance among the PCR variants containing multiple mutations. Four of these (T51, T53, D254, Y258) were chosen, and verified to be authentic sites of resistance by using site-directed mutagenesis to create single mutants; these amino acids reside within the active site and have been implicated in catalysis (1). An additional two residues (D116 and M219) were also shown by site-directed mutagenesis to yield resistance mutations (D116A and M219V, respectively.) The location in the three dimensional structure of all the single mutations analyzed here, together with the location of other previously identified resistance mutations, is shown in FIG. 4.

Discussion

In the course of investigations it was found that the resistant variants, listed in Table II, harbor substitutions throughout the linear sequence (FIG. 2) and three-dimensional structure (FIG. 4) of the protein. Some of the 74 different replacements (or combinations thereof) are responsible for 5-FdUR resistance, including the substitutions in single mutants, while other replacements are presumably co-selected and not relevant to resistance.

Recent crystallograhic analyses of TS, especially closed ternary complexes with dUMP and folate-based inhibitors, allowed to evaluate these replacements with a view toward understanding possible structure function relationships, and establishing fresh targets for directed mutagenesis. We have used the atomic coordinates of the wild type amino acids in tightly closed complexes with dUMP and a folate analog inhibitor [Protein Data Bank (NCBI, National Center for Biotechnology Information, U.S.A., GenBank® entries) 1HVY and 1JU6, respectively] to locate each amino acid replacement in the threedimensional structure, and to consider possible bases for resistance.

Single Mutants

The single mutants generated by PCR (see Table II) contain amino acid replacements that are distributed throughout the protein. Possible resistance mechanisms can be envisioned for some of the replacements, while no mechanisms are apparent for others. For example, substitution of valine for glutamate in the D48V mutant would disrupt the hydrogen bonding network within the Arg50-loop that mediates dUMP binding, and also affect interactions with the second subunit of the obligate homodimer; it has been proposed that mutations at residues 4752 may confer resistance by destabilizing the closed ternary complex. At position 84 in the V84A mutant, wild type valine interacts with two residues, Phe80 and Phe225, that contact the cofactor. Altered interactions with folate are known to confer 5-FdUR resistance, and could account for the resistance in the mutant. At position 110, wild type aspartate interacts with Trp109, which in turn interacts with both dUMP and cofactor, perhaps accounting for resistance of the D110E mutant. At position 194 in the active site, wild type proline interacts with the catalytic cysteine, presumably accounting for the resistance of the F194L mutant. At position 250, wild type histidine interacts with several residues that contact dUMP, including Asp226, presumptively accounting for the resistance of the H250L mutant. In contrast, resistance mechanisms for the single mutants E23G, K93E, S206G and K284N are not apparent. As shown in FIG. 4, E23G is in the disordered N-terminal region, while K93E, S206G and K284 are located in loops at the periphery of the TS monomer, and have no obvious relationship to catalysis or resistance. By using site-directed mutagenesis to create single mutants, we showed that several amino acids which were replaced in multiply mutated PCR variants are authentic sites of 5-FdUR. Among these site-directed replacements is T51S, the most common mutation previously observed and present in the highly resistant variant T51S, G52S. Thr51 participates in H-bonding within the Arg50-loop, and with Val313 after ligand-induced conformational changes of the C-terminal segment (1).

5-FdUR resistance could arise at Thr51 from several mechanisms, including an effect on this conformational change. Two replacements at Asp254 (D254N and D254E) were observed in multiply mutated variants (255, 318, 358), and we found that both, as well as the D254A substitution, conferred resistance as single mutations (FIG. 203C). Asp254 is H-bonded to His256, which is in turn H-bonded to dUMPbinding Arg175 in the second subunit; 5-FdUR resistance may arise from disruption of these interactions. We also observed that each replacement in the double mutant Y258S, T53S conferred resistance as a single mutation (FIG. 3B). Whereas the resistance of T53S may be attributable to disturbance of H-bonding in the Arg50-loop, the resistance of Y258F is likely due to loss of H-bonding between the tyrosine hydroxyl group and dUMP and/or Arg175′. The D116A substitution, like some of the PCR-generated single replacements, lies at the surface of the protein, and its contacts do not suggest why it confers resistance, Lastly, the D219V replacement affects Met219, which has multiple contacts with Tyr33, a known site of resistance, and with His256, which interacts with dUMP. Replacements in multiply mutated variants—The resistance arising from multiple substitutions is difficult to analyze. Nonetheless, evaluation of the replacements in the double and higher mutants listed in Table II and FIG. 2 can afford potentially useful inferences and point to particular amino acids for further examination, It is a logical assumption that regions where mutations cluster may have special relevance for resistance. One such region includes residues Val45 through Thr55 constituting the Arg50-loop. Wild type residues at the 6 mutated positions (45, 48, 51, 53, 55) participate in a network of interactions, primarily H-bonding, within the loop, and also between the Arg50-loop and the second subunit that contains dUMP-binding residues 175′ and 176′. Replacements at these 6 positions might affect interactions with both dUMP and 5FdUMP, and also destabilize the ternary complex, thereby conferring resistance. A conspicuous cluster of mutations was noted at positions Arg78 through Leu88, where 7 residues harbor 9 different replacements. Phe80 in this region interacts with the co-factor, while Glu87 may have destabilizing interactions with both the co-factor and dUMP. Wild type amino acids at four mutated positions (78, 81, 82, 84) contact Phe80, while those at three mutated positions (84, 85, 83) interact with Glu87. Altered interactions with folate and/or dUMP may account for the resistance conferred by replacements in this region. Of particular interest with respect to folate binding is the occurrence of the M311L replacement in the double mutant 217. Met311, together with three wild type residues that yield 5-FdUR resistant mutants, form a hydrophobic collar around the paminobenzoic acid ring in the folate analog in a new ternary complex. A third group of 14 replacements was observed in the N-terminal 27 amino acids, which are disordered in crystal structures (Carreras and Santi, 1995). This observation raises the possibility that at least some wild type amino acids in the N-terminal region may have an unrecognized function(s) in 5-FdUR resistance, a possibility substantiated by identification of the single mutant E23G. A related possibility is that the N-terminus may have a specific function(s) and an ordered structure(s) in conformational states which have not been captured in existing crystals. Another possible source of 5-FdUR resistance in multiply mutated variants is altered interactions between the subunits of the obligate TS homodimer; these interactions may be important for positioning of dUMP-binding residues in one or both subunits, and may thus affect 5-FdUR resistance. For example, Val45 in the Arg50-loop interacts with V204 in the second subunit, and we have previously isolated a 5-FdUR-resistant V204D mutant. The Val204Val45 hydrophobic interaction was disrupted in four different multiply substituted mutants isolated here, two involving Val45 (mutants 255 and 326) and two involving Val 204 (mutants 316 and 359). In another example of subunit interactions, Glu211 resides at the dimer interface where it contacts Phe59 from the second subunit. We have previously identified the 5-FdUR resistance mutation Q211 L in a single mutant, and isolated it again here in the triple mutant 313. All told, we can offer a plausible structure-based suggestion for the resistance of most of the 35 mutants in Table II, though only 5 of 29 contain a single substitution. These mutants contain a replacement at a position where the wild type residue is either: (a) In direct contact with dUMP or a residue that contacts dUMP; and/or (b) in direct contact with folate or a residue that contacts folate; and/or (c) interacts with the second subunit at the dimer interface. We and others have previously documented 5-FdUR resistance mutations in these three categories. We rationalize our observations concerning resistance mutations at ligand-interacting residues as follows.

Wild type amino acids that interact directly with substrates or co-factors are likely to be important for catalytic activity, and to yield relatively few replacements that conserve catalytic efficiency, alter substrate/inhibitor preference and afford resistance. This relative lack of mutability not with standing, drug resistance mutations at such residues could be identified by intensive, targeted mutagenesis. On the other hand, residues that contact ligand-interacting residues may yield a relatively large number of substitutions that simultaneously preserve activity, modulate substrate/inhibitor interactions and confer drug resistance; mutations at such residues may be highly prevalent in the present work, where each residue in the protein has a limited probability of being substituted. We can also suggest possible sources of 5-FdUR resistance for the four single amino acid replacements that are far removed from the active site in current crystal structures. We have discussed the E23G mutation in the disordered, N-terminal segment, and noted that three others (K93E, S206G and K284) are located in loops at the surface of the monomer (FIG. 4). If TS functions in a multiprotein complex in vivo, these loops may be involved in protein-protein interactions that after the conformation of TS and affect resistance indirectly. It is also possible that the loops are involved in the conformational dynamics of the protein, and affect motions that influence the probability of reactions at the active site. Studies of single enzyme molecules by confocal fluorescence spectroscopy indicate that enzymes undergo sets of transient oscillations during each catalytic step. Amino acid substitutions at a distance may affect these oscillations, and thereby alter substrate and inhibitor discrimination at the active site. Perhaps yet to be determined mechanisms that govern conformational changes during catalysis can be affected by amino acid replacements throughout the protein.

### Example 2

### Medicament for Locally Treating or Preventing Chemothrerapy Induced Mucositis and Other

Expression and Isolation of PTD-4-TS_{D254E}-Fusion Protein

The vector pTAT-HA, which has been already described (Nagahara et al, 1998) was used for the expression of the PTD-4-TS_{D2S4E}-fusion protein. The original TAT sequence (YGRKKRRQRRR, SEQ ID NO: 5) was exchanged in this vector by a TAT sequence (YARAAARQARA, SEQ ID NO: 6), which was optimized by random mutagenesis. The optimized TAT sequence has a 33 fold more efficient transduction activity in comparison to the original TAT sequence and will be designated as PTD-4 (“protein transduction domain 4”) in the following (Ho et al., 2001). Into the vector pTAT-HA, which was modified in this way, the sequence of wild type thymidylate synthase TS_{wt} and of the TS mutant TS_{D254E}, respectively, were inserted under maintenance of their reading frame. The vector causes the expression of fusion proteins with 6 histidine residues N-terminal to the TAT-sequence. TAT-TS-fusion proteins were generated by transfection of BL2 1 (DE3)pLysS E. coli cells (Novagen, Madison, Wis., USA) with this vector. For this purpose bacteria were cultured over night and the fresh culture was inoculated and incubated for 4 to 6 hours at 37° C. in a shaker incubator. Cells were separated from the medium by centrifugation and the cell pellet was lyzed with 10 mL buffer A (8 M urea, 20 mM HEPES, pH 8.0 and 100 mM sodium chloride). Unsoluble cell debris was separated by centrifugation und the clear supernatant was applied on to a Ni-NTA nickel chelate chromatography column (Qiagen, Valecia, Calif., USA). The column was washed with buffer A supplemented with 10 mM imidazol and the bound protein was eluted from the column in three steps with buffer A, which was supplemented with 100, 250, and 500 mM imidazol, respectively. The fractions, which contained the TAT-TS-fusion protein, were diluted 1:1 with a buffer, which contained 20 mM HEPES resulting in the following buffer composition: 4 M urea, 20 mM HEPES, pH 8.0, and 50 mM sodium chloride. The sample was applied on to a Mono Q 10/10 chromatography column (Pharmacia, Piscataway, N.J., USA) using a FPLC-chromatography device. Subsequently the column was rinsed with the same buffer, which did not contain urea, i.e., with 20 mM HEPES, 50 mM sodium chloride, pH 8.0 and the bound TAT-TS-fusion protein was eluted from the column with a buffer, which contained 1 M sodium chloride, 20 mM HEPES, pH 8.0. These purified fusion proteins were adjusted to a PBS-buffer by means of gel filtration with a PD10 sephadex G-25 gel filtration column (Pharmacia, Piscataway, N.J., USA), which determines the protein concentration with a Bradford protein assay in a way known to the skilled person. Then the protein was shock frozen with liquid nitrogen and stored at −80° C.

Testing the 5-FU-resistance of TS-Mutants

The bacterial strain χ2913 was transfected with expressions vectors of the TS-mutants and defined amounts of bacteria in the exponential growth phase were plated on selection plates. The selection plates contained increasing concentrations of 5-fluorodeoxyuridine (0, 75, 150, 200, respectively 300 nM). 5-fluorodeoxyuridine (5-FdUR) is a very effective metabolite, which is generated from 5-fluorouracil in the cell. The selection plates were made of minimal medium, which contained carbenicilline and tetracycline. Since the selection plates did not contain thymidine, only those bacteria can grow, which have a functioning thymidylate synthesis that is not inhibited by the concentration of the TS-inhibitor 5-FdUR present in the plates. After 36 to 48 hours the number of growing colonies was determined. For this the number of colonies of bacteria, which were transfected with wild type thymidylate synthase was set as 100% and all other numbers of colonies were correspondingly converted to % values.

Treatment of 3T3-Fibroblast Cell Line with 5-FU and TAT-TS-Fusions Proteins

3T3 fibroblasts cells (available by the American Type Cell Collection (ATCC, Rockville, Md., USA) were seeded with a cell density of 1×10⁴ cells per well into 96-well-cell culture plates. DMEM medium with 10% fetal calve serum is used as cell culture medium, and cells are cultured in a CO₂-incubator at 37° C. and 5% CO₂. The cell culture medium is aspirated after 24 hours and substituted by 200 μL/well of the test assay and cells are incubated for another 24 hours. Subsequently the portion of viable cells was determined as described below. The test assays are composed as follows:

Controls without 5-FU and without PTD-4-TS_{D2S4E}-fusion protein: 200 μL/well of DMEM with 10%.fetal calve serum

Samples with 5-FU without PTD-4-TS_{D2S4E}-fusion protein: 200 μL/well of DMEM with 10% fetal calve serum and 2.5 μM 5-FU

Samples with 5-FU and PTD-4-TS_{D2S4E}-fusion protein (wild type TS or TS-mutants): 200 μL/well of DMEM with 10% fetal calve serum, 2.5 μM 5-FU and 40 ng!mL PTD-4-TS_{D2S4E}-fusion protein

The fusion protein PTD-4-TS_{D254E} was made as described above.

Determination of the Cytotoxicity of 5-FU in Fibroblasts in the Presence or Absence of TAT-TS-Fusion Proteins with the Dye Hoechst 33342

After completion of the 24-hour treatment of the cells in the control- or test assaysas described above, the medium is removed from the 96-well-plates and substituted by 200 μL/well DMEM medium with 10% fetal calve serum and 10 μg/mL of the DNA-binding fluorescent dye H33342 (Engelmann et al., 2001). The plates are kept at 37° C. for 30 minutes and are then washed twice with each 100 μL/well phosphaste-buffered sodium chloride solution (PBS, “phosphate buffered saline”). Subsequently, 100 μL well PBS is added and the fluorescence is quantified at an excitation wave length of 460 nm and an emission wave length of 360 nm. The intensity of the fluorescence signal is a measure for the number of viable cells. The fluorescence background signal (dye incubation without cells) is subtracted from the raw data of the fluorescence readings and the calculation of the %-share of viable cells is made related to the fluorescence reading of cells, which were not treated with 5-FU. The fluorescence intensity of these samples is set as 100%. The dosage of 5-FU that kills 50% of the cells (ED₅₀), is determined using the same method. These experiments resulted in an ED₅₀ of 2.5 μM 5-FU for the 3T3-fibroblast cell line and an ED₅₀ of 10 mM 5-FU for human primary gingival fibroblasts. The ED₅₀-values were calculated using the nonlinear regression. This method to determine the ED₅₀ has been already described (Engelmann et al., 2001).

Isolation of Primary Human Gingival Fibroblasts

Primary human gingival fibroblasts were obtained from gingival tissue that arises during the extraction of wisdom teeth of sound persons. This gingival tissue was cut into 1 mm² sized pieces and was then stored over night in DMEM with 10% fetal calve serum and antibiotics at 4° C. Thereafter, the tissue samples were transferred into fresh cell culture dishes, slightly dried for a better adhesion to the cell culture plates, and cultured in DMEM with 10% fetal calve serum at 37° C. and 5% CO₂ Gingival fibroblasts are grown out of the tissue pieces after 2 to 4 days and can then be further cultured and used for the following experiments.

Results

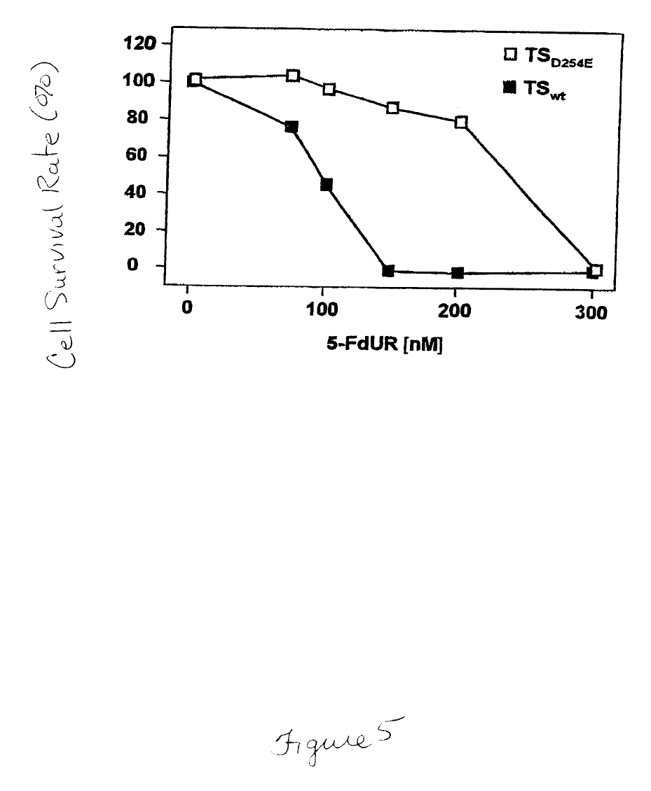

The results are depicted in FIGS. 5-7.

As can be seen in FIG. 5, 80% of the TS mutants TS_{D254E} survive even at 200 nM 5-FdUR whereas no wild type TS_{WT} survived at this 5-FdUR concentration.

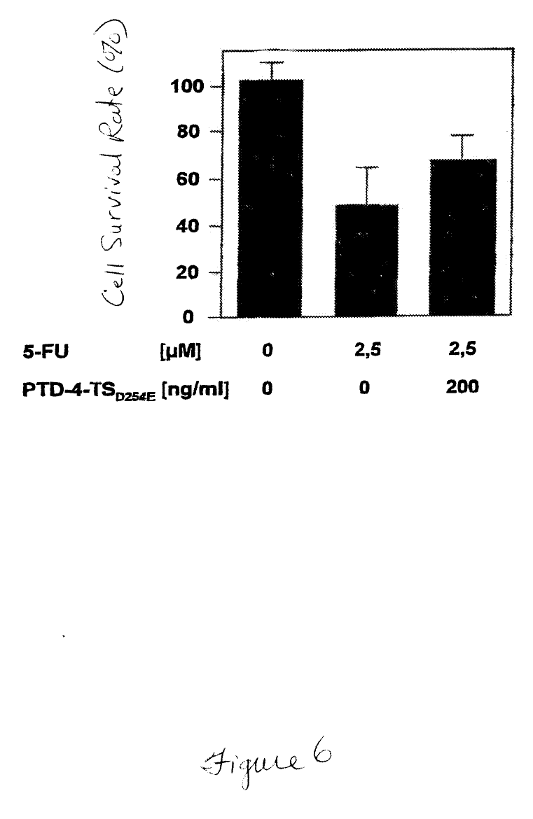

As can be seen in FIG. 6, since 2.5 μM 5-FU represent the ED₅₀ concentration of this drug for 3T3 cells, only 50% of the cells survived in the absence of PTD-4-TS_{D254E} (2nd column from left). However, 70% of the cells, which were also treated with PTD-4-TS_{D254E}, survived the chemotherapy with 5-FU (3. column from left). The left column shows the control, 100% of the 3T3 cells, which were not treated with 5-FU survived.

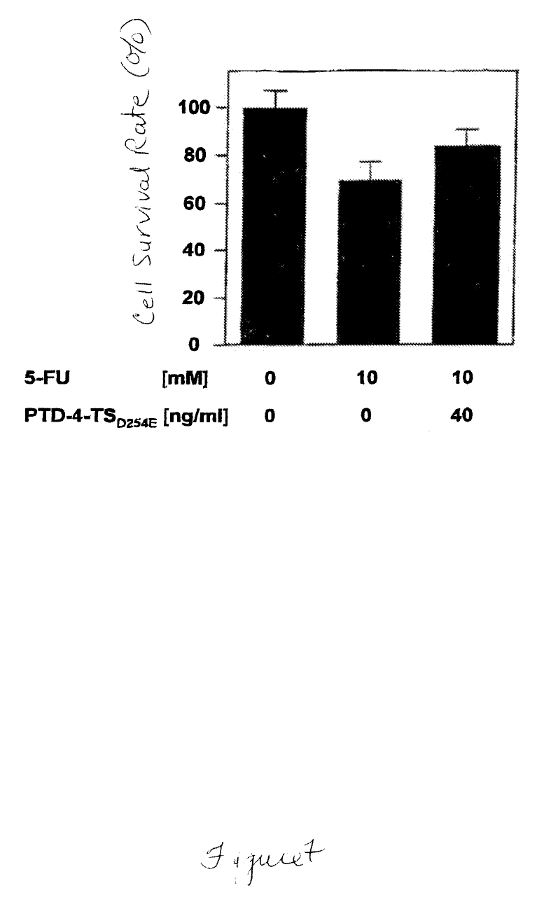

As can be seen in FIG. 7, Primary gingival fibroblasts reveal a significantly higher ED₅₀ for 5-FU compared to the faster growing 3T3 cells due to their slower proliferation. 85% of the gingival fibroblasts, which were treated with PTD-4-TS_{D2S4E} survived the chemotherapy with 5-FU (3rd column from left), whereas only 70% of the untreated gingival fibroblasts survived. The left column shows the control; 100% of gingival fibroblasts, which were not treated with 5-FU, survived.

### REFERENCES

Ahmad, S. I., Kirk, S. H., and Eisensterk, A. (1998) Annu. Rev. Microbiol, 52, 591625

Barsoum J G, Fawell S E, Pepinsky R B. 1992. TAT-derived transport polypeptides. In WO 94/04686. Biogen Inc, Cambridge Mass., USA, 155 pp.

Bertino J R, Tong Y, Liu-Chen X, Banerjee D. 1997. Mutants of thymidylate synthase and uses thereof. In WO 98/33518. Sloan-Kettering Institute for Cancer Research, New York, N.Y., USA 78pp

Carreras C W, Santi D V. 1995. The catalytic mechanism and structure of thymidylate synthase. Annu Rev Biochem. 64: 721-762

Chariot: the vehicle of the future for protein transfection. Active Motif, 2001. Carlsbad, Calif., USA, 15 pp

Danenberg, P. V., Malli, H., and Swenson, S. (1999) Semin. Oncol. 26, 621631

Dowdy S F. 2000. Protein transduction: delivery of TAT-fusion proteins into mammalian cells. Laboratory manual SFD-Lab.

Engelmann J, Leyhausen G, Leibfritz D, Geurtsen W. 2001. Metabolic effects of dental resin components in vitro detected by NMR spectroscopy. J Dent Res. 80: 869-875.

Ho A, Schwarze S R, Mermelstein S J, Waksman G, Dowdy S F. 2001. Synthetic protein transduction domains: enhanced transduction potential in vitro and in vivo. Cancer Res. 61: 474-477

Nagahara H, Vocero-Akbani A M, Snyder E L, Ho A, Tatham D G, Lissy N A, Becker-Hapak M, Ezhevsky S A, Dowdy S F. 1998. Transduction of full-length TAT fusion proteins into mammalian cells: TAT-p27Kip1 induces cell migration. Nat Med. 4: 1449- 1452 Protein Data Bank NCBI, National Center for Biotechnology Information, U.S.A. (GenBank®); world wide web: wvwi.ncbi.nlm.nih.gov: Einträge: 1 JUJA. Chain, A., Human Thymidylate Synthase; P04818 TYSY_Human; XP_{—}046765 Thymidylate synthase homo sapiens, BAB 83676 Thymidylate Synthase home sapiens

Rustum, Y. M., Harstrick, A., Ceo, S., Vanhoefer, U., Yin, M. B., Wilke, H., and Seeber, S. (1997) J. Clin. Oncol. 15, 389400

Schwarze S R, Dowdy S F. 2000. In vivo protein transduction: intracellular delivery of biologically active proteins, compounds and DNA. Trends Pharmacol Sci. 21: 45-48

Tong Y, Liu-Chen X, Ercikan.-Abali E A, Capiaux G M, Zhao, Banerjee D, Bertino J R 1998. Isolation and characterization of thymitaq (AG337) and 5-fluoro-2deoxyuridylate-resistant mutants of human thymidylate synthase from ethyl methanesulfonate-exposed human cells. J Biol Chem. 273: 11611-11618

1. Recombinant cDNA encoding a thymidylate synthase (TS) mutant, wherein said recombinant cDNA encodes a TS mutant protein that differs from human wildtype TS at four positions as follows: T51S, K82Q, K99D, and N171S.

2. A DNA vector comprising one or more copies of a recombinant cDNA encoding a TS mutant, wherein said recombinant cDNA encodes a TS mutant protein that differs from human wildtype TS at four positions as follows: T51S, K82Q, K99D, and N171S.

3. A TS mutant protein, wherein said TS mutant protein differs from human wildtype TS at four positions as follows: T51S, K82Q, K99D, and N171S.

4. A fusion protein comprising
a transductor for the transduction of a protein into a human cell; and,
a TS mutant protein, wherein said TS mutant protein differs from human wildtype TS at four positions as follows: T51S, K82Q, K99D, and N171S.

5. A host cell comprising a nucleic acid encoding a TS mutant protein, wherein said TS mutant protein differs from human wildtype TS at four positions as follows: T51S, K82Q, K99D, and N171S.

6. A pharmaceutical composition comprising
a TS mutant protein, wherein said TS mutant protein differs from human wildtype TS at four positions as follows: T51S, K82Q, K99D, and N171S; or
a nucleic acid encoding a TS mutant protein, wherein said TS mutant protein differs from human wildtype TS at four positions as follows: T51S, K82Q, K99D, and N171S; and
a pharmacologically acceptable carrier.

7. A method of protecting cells against toxic effects of analogs that inhibit TS, comprising the steps of
providing said cells with a TS mutant protein, wherein said mutant TS protein is resistant to said toxic effects, and wherein said TS mutant protein differs from human wildtype TS at four positions as follows: T51S, K82Q, K99D, and N171S.

8. The method of claim 7, wherein said step of providing is carried out by transfecting said cells with a nucleic acid molecule encoding said TS mutant protein.

9. The method of claim 7, wherein said toxic effects include side effects of chemotherapy.

10. The pharmaceutical composition of claim 6, wherein said pharmaceutical composition is prepared as an oral rinsing solution.
